(19) United States
(12) Patent Application Publication
(10) Pub. No.: US 2006/0131197 A1
(43) Pub. Date: Jun. 22, 2006

(54) Disposable package with a repositioning attachment feature

(75) Inventor: Cindy Lou Price, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/013,905

(22) Filed: Dec. 16, 2004

(51) Int. Cl. A61B 19/02

(52) U.S. Cl. 206/440

(57) ABSTRACT

An article storage system (80) comprises a package (82), a plurality of personal care articles (20) contained in the package, and a mounting mechanism (46) which is configured to releasably attach the package (82) to an appointed mounting surface. Each article (20) may, for example, include a liquid-permeable topsheet (26) and a backsheet (28). In a desired aspect, the backsheet can be operatively liquid-impermeable. The mounting mechanism (46) is secured to the package (82). In a desired aspect, the mounting mechanism (46) may be substantially permanently affixed to an outwardly-facing surface of the package. The mounting mechanism (46) can also include first and second, distal, lateral portions (48) which are laterally spaced apart by a selected spacing distance (50). In particular aspects, the package can be flexible and collapsible, and the mounting mechanism (46) can include a distinctive vertical distribution. The package (82) can also include an exit mechanism (84) which is configured to provide a package opening (86) through which the articles (20) can be removed from the package (82).

### FIELD OF THE INVENTION

The present invention relates to a system for holding selected articles. More particularly, the present invention can provide a system for storing and/or dispensing personal care articles, and the articles may be absorbent articles, such as feminine care or adult incontinence products.

### BACKGROUND OF THE INVENTION

Personal care articles, particularly personal care absorbent articles are well known in the art. The absorbent articles have been intended to absorb discharged body fluids, such as urine and/or menses. Such articles and products generally comprise a liquid permeable topsheet and a backsheet. Additionally, a fibrous mass or other absorbent body, which can absorb and hold the body fluids, is assembled between the topsheet and backsheet. Incontinence products have typically been employed to absorb liquids, such as urine. Feminine care articles have typically been employed to absorb urine, menses and other vaginal discharges. In particular arrangements, the feminine care articles have included a conventional garment-fastener for securing an individual article in a wearer's undergarment. In further arrangements, the articles have included wing portions which can help to hold the article in place at a selected location in a wearer's undergarment. In some arrangements, the wing portions have been integrally formed with one or more of the preexisting component layers that were employed to construct the article. In other arrangements, the wing portions have been separately provided components that are assembled and affixed to the final product. Conventional wing-fasteners have been employed to secure the wing portions in a desired configuration during ordinary use. The various garment-fasteners and wing-fasteners have included conventional fastening mechanisms, such as adhesive fasteners and mechanical fasteners. The mechanical fasteners have typically included conventional, hook-and-loop fasteners.

The personal care articles have also been contained in various conventional packaging systems. Individual articles have included wrappers, such as individual containment pouches composed of polymer films and/or nonwoven fabrics. Predetermined quantities of articles have also been grouped and contained in conventional packages, such as bags and/or cartons, and the selected packages could be opened to allow a desired extraction of individual articles. Systems employing mounting brackets, mounting rails, and hook-and-loop type fasteners have been employed to mount and hold containers and implements in a suspended position.

Conventional packaging and mounting systems, however, have not provided desired combinations of convenience and easy access to the individual articles within a package. The packaging systems have employed complex structures, and have been overly expensive to manufacture. The systems and structures have also been cumbersome to use, and have not been configured for convenience placement at desired locations. Conventional packaging and mounting systems have continued to suffer from excessive collapse before the packaging has been emptied of product, making it difficult to find and extract individual products. Additionally, conventional packaging and mounting systems have been susceptible to an excessive shifting or movement within the storage area causing loss of time searching for the package, and have been susceptible to excessive spillage. Conventional mounting systems have taken excessive time to set up or have caused undesired damage to mounting surfaces. It has, for example, remained difficult to provide a packaging system that is inexpensive, easy to use, and does not occupy excessive amounts of space on a shelf, countertop or wall surface. As a result, there has been a continued need for an improved packaging and dispensing system that can be efficiently manufactured and provide a more convenient storage and access to the packaged articles.

### BRIEF DESCRIPTION OF THE INVENTION

Generally stated, the present invention can provide an article storage system which comprises a package, a plurality of personal care articles contained in the package, and a mounting mechanism which is configured to releasably attach the package to a mounting surface. In a desired aspect, the support surface can be a generally vertical surface. The mounting mechanism is operatively secured to the package, and in a particular aspect, the mounting mechanism can be substantially permanently affixed to an outwardly-facing surface of the package. In another aspect, the mounting mechanism can be secured to the package with a factory-attachment. The mounting mechanism can also include first and second, distal, lateral portions which are laterally spaced apart by a selected spacing distance. The spacing distance can be at least a minimum percentage of an overall width of the package, as determined when the package is filled with the articles. In a particular aspect, a distinctive vertical distribution, and in another aspect, the operative portion of the mounting mechanism can include a distinctive aspect ratio. The package can further include an exit mechanism configured to provide a package opening through which the articles can be removed from the package.

By incorporating its various aspects, features and configurations, the storage system of the invention can more effectively provide a convenient means of storing and presenting a product package so that the package can remain upright and the package opening can stay relatively open for easy access to the contained articles. The storage system of the invention can operatively secure the package at a desired location, and can allow an easy transfer of the package to another location, as desired. Additionally, the package can be readily hung, suspended from or otherwise operatively attached to a vertical surface, and then selectively removed from the surface when the package has been emptied of its contained articles. The package can be placed in more convenient locations, such as on cabinet doors, and can be positioned without occupying any shelf space needed for other items. The storage system can more effectively resist collapse before the package has been emptied of absorbent articles, and help to improve the ease of finding and removing desired articles. The storage system can better resist excessive movement and can better resist an excessive spilling of products. Additionally, the storage system can be quickly mounted and can be repositioned without excessively damaging the previously employed mounting surface.

### BRIEF DESCRIPTION OF THE DRAWINGS

The various features, aspects and advantages of the present invention will become better understood with reference to the following description, appended claims and accompanying drawings where:

FIG. 1 shows a schematic, partially cut-away view of a representative system for retaining articles.

FIG. 1A shows a schematic view of a representative mounting panel and mounting mechanism in a system for retaining articles.

FIG. 2 shows a partially cut away, top plan view of a bodyside of a representative personal care article.

FIG. 3 shows a view of a representative, longitudinal cross-section through a personal care article of the invention.

FIG. 4 shows a partially cut away, top plan view of a garment-side of a representative feminine care article having a pair of wing-panels, where the wing-panels are arranged in a laterally-extended position.

FIG. 5 shows a schematic view of a representative, article storage system which includes a mounting mechanism having horizontally and vertically elongated portions.

FIG. 5A shows a schematic view of a representative article storage system which includes a mounting mechanism having vertically aligned and vertically elongated portions.

FIG. 5B shows a schematic view of another representative article storage system which includes a mounting mechanism having generally, parallel, vertically elongated sections.

FIG. 5C shows a schematic view of a representative article storage system which includes a mounting mechanism having an array of generally parallel, horizontally elongated sections, and generally parallel, vertically elongated sections.

FIG. 5D shows a schematic view of a representative article storage system which includes a mounting mechanism having another array of vertically elongated sections.

FIG. 5E shows a schematic view of a representative article storage system which includes a mounting mechanism having a plurality of vertically oriented strips.

FIG. 5F shows a schematic view of a representative article storage system which includes a mounting mechanism having a curved mounting mechanism.

FIG. 5G shows a schematic view of a representative article storage system which includes a mounting mechanism distributed in a discontinuous array.

FIG. 5H shows a schematic view of a representative article storage system which includes a mounting mechanism distributed in an array of graphics.

FIG. 5I shows a schematic view of a representative article storage system which includes a mounting mechanism distributed in an array of text.

FIG. 6 representatively shows a schematic view of an article storage system which includes a handle member.

FIG. 6A shows a schematic view of the storage system of FIG. 6, in which the handle member is positioned to expose the mounting mechanism for use.

FIG. 7 representatively shows a schematic view of an article storage system which includes a system of pleats.

FIG. 7A shows a schematic view of the storage system of FIG. 7, in which the pleats are expanded and positioned to expose the mounting mechanism for use.

FIG. 8 representatively shows a schematic view of an article storage system which locates a portion of the mounting mechanism on a flap section.

FIG. 8A representatively shows a schematic view of the storage system of FIG. 6, in which the flap section is in an open position and operatively attached to the appointed support surface.

### DETAILED DESCRIPTION OF THE INVENTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

By the terms “particle,” “particles,” “particulate,” “particulates” and the like, it is meant that the material is generally in the form of discrete units. The units can comprise granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large, greatest-dimension/smallest dimension ratio, like needles, flakes and fibers are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

As used herein, the term “nonwoven” refers to a fabric web that has a structure of individual fibers or filaments which are interlaid, but not in an identifiable repeating manner.

As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

“Coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein, the phrase “complex liquid” describes a liquid generally characterized as being a viscoelastic liquid comprising multiple components having inhomogeneous physical and/or chemical properties. It is the inhomogeneous properties of the multiple components that challenge the efficacy of an adsorbent material in the handling of complex liquids. In contrast with complex liquids, simple liquids, such as, for example, urine, physiological saline, water and the like are generally characterized as being relatively low-viscosity and comprising one or more components having homogeneous physical and/or chemical properties. As a result of having homogeneous properties, the one or more components of simple liquids behave substantially similarly during absorption or adsorption.

Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells.

As used herein, the phrase “absorbent article” refers to devices which absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, tampons, interlabial devices and the like), infant diapers, children's training pants, adult incontinence products and the like; as well as absorbent wipes and covering mats.

Disposable absorbent articles such as, for example, many of the feminine care absorbent products, can include a liquid pervious topsheet and a backsheet operatively connected to the topsheet. Additionally, the article may include an absorbent core positioned and held between the topsheet and the backsheet. The topsheet is operatively permeable to the liquids that are intended to be held or stored by the absorbent article, and the backsheet may be substantially impermeable or otherwise operatively impermeable to the intended liquids. The absorbent article may also include other components, such as liquid wicking layers, liquid intake layers, liquid distribution layers, transfer layers, barrier layers, and the like, as well as combinations thereof. Disposable absorbent articles and the components thereof can operate to provide a body-facing surface and a garment-facing surface. As used herein, “body-facing surface” means that surface of the article or component which is intended to be disposed toward or placed adjacent to the body of the wearer during ordinary use, while the “outward surface” or “outward-facing surface” is on the opposite side, and is intended to be disposed to face away from the wearer's body during ordinary use. The outward surface may be arranged to face toward or placed adjacent to the wearer's undergarments when the absorbent article is worn.

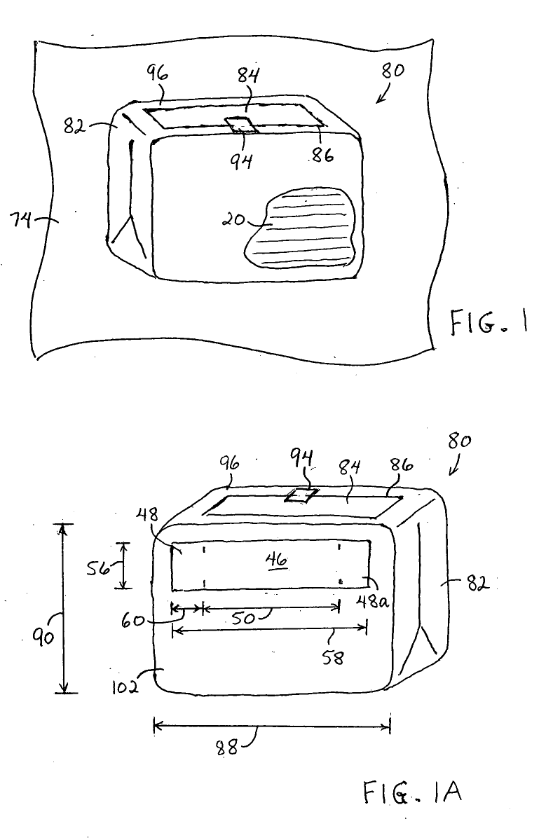

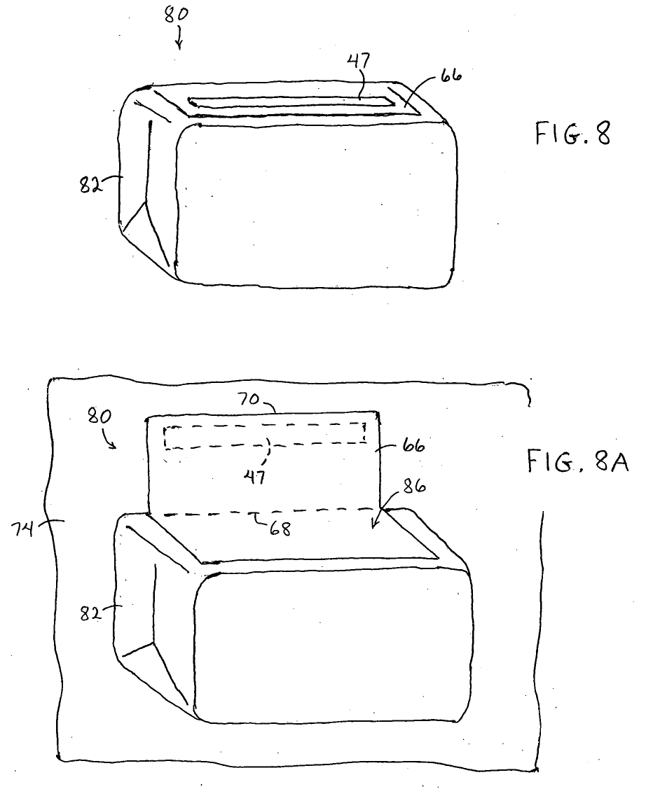

With reference to FIGS. 1 through 4, the present invention can provide an article storage system 80 which comprises a package 82, a plurality of articles 20 contained in the package, and a mounting mechanism 46 which is configured to releasably attach the package 82 to a support surface. In a desired aspect, the support surface can be a generally vertical surface. Each article 20 can be a personal care article, and in desired arrangements, each article may include a liquid-permeable topsheet 26 and a backsheet 28. In a desired aspect, the backsheet can be operatively liquid-impermeable. The mounting mechanism 46 is operatively secured to the package 82. In a particular aspect, the mounting mechanism 46 can be integrated and substantially permanently affixed to an outwardly-facing surface of the package. In other aspects, the mounting mechanism 46 can be affixed or otherwise operatively secured to the package 82 with a factory-attachment, and the mounting-mechanism may be operatively secured to an outer surface of the package. The mounting mechanism 46 can also include first and second, distal, lateral portions 48 which are laterally spaced apart by a selected spacing distance 50. The spacing distance 50 can be at least a minimum percentage of an overall width 88 of the package 82, as determined when the package is filled with the articles 20. In a particular aspect, the spacing distance can be at least a minimum of about 5% of the overall width 88 of the filled package 82. In another aspect, at least a significant, operative portion of the mounting mechanism 46 can include a distinctive vertical aspect ratio. The package 82 can further include an exit mechanism 84 which is configured to provide a package opening 86 (e.g. FIG. 8A) through which individual or groups of articles 20 can be removed from the package 82. Still other features of the invention are set forth in the present disclosure. The storage system 80 can desirably be a limited use system, and the storage system can be discarded after the system has been emptied of articles.

By incorporating its various aspects, features and configurations, the storage system of the invention can more effectively provide a convenient means of storing and presenting a product package so that the package can remain upright and the package opening can be readily opened for easy access to the contained articles. The storage system of the invention can operatively secure the package at a selected initial location, and can allow an easy transfer of the package to another location, as desired. The package can be hung or otherwise suspended from a selected vertical surface, and selectively removed from the surface when the package has been emptied of its contained articles. The securing of the package in a substantially upright position can allow an easier access into the package for a longer period of time. Desirably, the ready access can be maintained for substantially the entire dispensing period. The package opening can more readily remain open, or can be more readily moved to the desired open position when the user desires access into the package. The mounting of the package to an operatively firm, support surface can help maintain the package shape and dimensions. The storage system can more effectively resist collapse before the package has been emptied of absorbent articles, and help to improve the ease of finding and removing desired articles. Additionally, the package can be placed in more convenient locations, such as on cabinet doors, and can be positioned without occupying shelf space needed for other items. The storage system can better resist excessive movement and can better resist an excessive spilling of products. Additionally, the storage system can be quickly mounted and can be repositioned without excessively damaging the previously employed mounting surface.

Various articles, such as the shown personal care article, can be suitably configured for inclusion in the present invention. The personal care article can typically be configured for prolonged, substantially continuous contact against the skin or other external or internal surfaces of a wearer's body. In desired arrangements, the personal care article can be a diaper, an adult incontinence article, an article of disposable underwear, a disposable undergarment, a feminine care article, or the like. The feminine care article can, for example, be a feminine care tampon, pad, napkin, pantiliner or the like. The article may or may not include an absorbent.

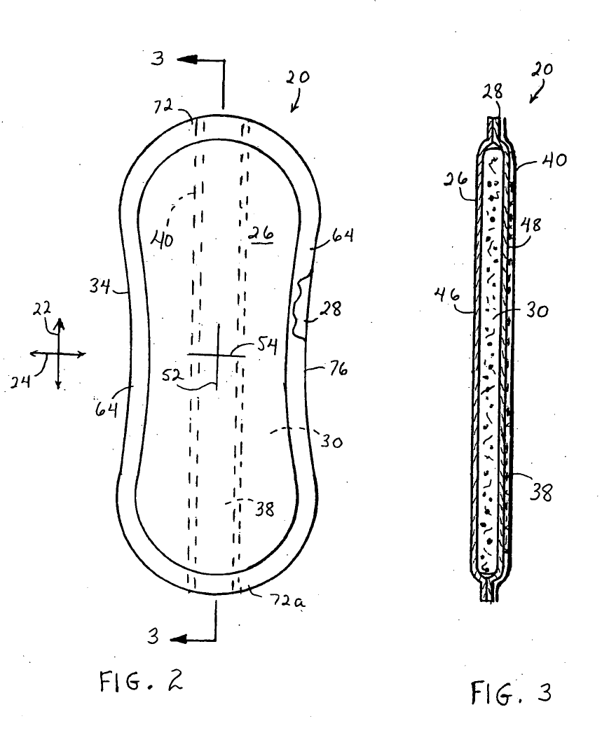

FIGS. 2 through 4 show representative examples of a suitable article 20. In desired arrangements, the personal care article 20 can have an appointed bodyside surface, an appointed garment-side surface, a lengthwise longitudinal-direction 22, a lateral cross-direction 24, a pair of longitudinally-extending, laterally-opposed side edge regions 64, and an intermediate portion 76 which is interposed between a pair of longitudinally opposed end portions 72 (72, 72a). The article also has a longitudinally extending centerline 52 and a laterally extending centerline 54. As representatively shown, the longitudinal dimension of the article is relatively larger than the lateral dimension of the article. In particular configurations, the article 20 can include a baffle or backsheet 28, and a liquid-permeable cover or topsheet 26 which is operatively connected in a facing relation with the backsheet. In particular configurations, the article may further include an absorbent body structure 30 which is operatively positioned and sandwiched between the backsheet 28 and topsheet 26.

As representatively shown, peripheries of the topsheet and backsheet may be substantially entirely coterminous. Alternatively, the peripheries of the topsheet 26 and the backsheet 28 may be partially or entirely non-coterminous. Each article 20 can desirably have a shape which is nonrectangular. In a particular feature, the article has a perimeter terminal edge 34, and a significant portion of the terminal edge 34 can be curvilinear.

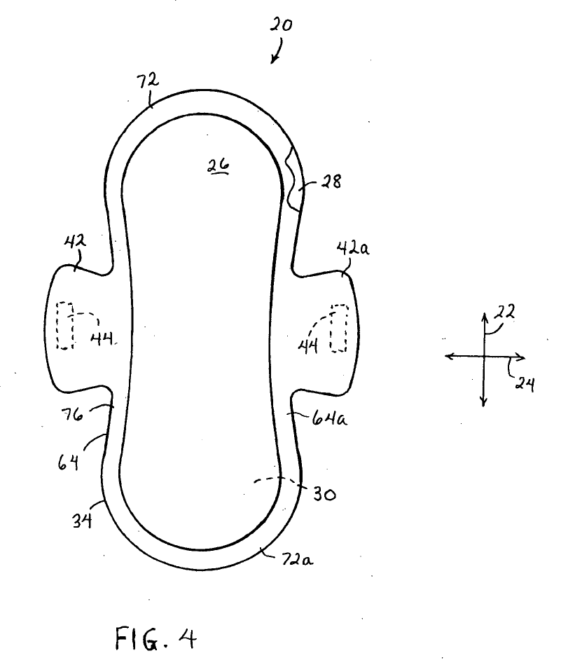

In a desired feature, the article can also include at least one pair of wing-panels 42 (e.g. FIG. 4). A first wing-panel 42 can be attached to a first side edge region 64 in the intermediate portion 76 of the article 20, and a second wing-panel 42a can be attached to a second side edge region 64a in the intermediate portion 76 of the article 20. Each wing-panel 42 (42, 42a) can be configured to wrap about an undergarment of a wearer. A panel fastener 44 can be operatively joined or otherwise operatively connected to each wing-panel 42, and each panel fastener 44 can be configured to operatively secure its corresponding wing-panel 42 about the undergarment of the wearer during ordinary use. Each wing-panel 42 can include a panel base section and a panel free-end region, and each panel base section can be operatively joined or otherwise operatively connected to its corresponding side edge region 64 of the article 20. Each panel free-end region can extend from its corresponding panel base section, and can be configured to operatively wrap around an appointed region of the wearer's undergarment. For example, the free-end regions of the wing-panels can be wrapped about a crotch region of the wearer's undergarment.

The cover or topsheet 26 may include any material that can be configured to provide the topsheet with an operative level of liquid-permeability. The topsheet may be constructed with one or more layers of suitable materials, and may be a composite material. For example, the topsheet layer can include a woven fabric, a nonwoven fabric, a polymer film, a film-fabric laminate or the like, as well as combinations thereof. Examples of a nonwoven fabric include spunbond fabric, meltblown fabric, coform fabric, a carded web, a bonded-carded-web, a bicomponent spunbond fabric or the like as well as combinations thereof. For example, the topsheet layer can include a woven fabric, a nonwoven fabric, a polymeric film that has been configured to be operatively liquid-permeable, or the like, as well as combinations thereof. Other examples of suitable materials for constructing the topsheet layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable topsheet layer material can include a bonded-carded-web composed of polypropylene and polyethylene, such as has been used as a topsheet stock for KOTEX brand pantiliners. Other examples of suitable materials are composite materials of a polymer and a nonwoven fabric material. The composite materials are typically in the form of integral sheets generally formed by the extrusion of a polymer onto a web of spunbond material. In a desired arrangement, the topsheet layer 26 can be configured to be operatively liquid-permeable with regard to the liquids that the article is intended to absorb or otherwise handle. The operative liquid-permeability may, for example be provided by a plurality of pores, perforations, apertures or other openings, as well as combinations thereof, that are present or formed in the topsheet layer. The apertures or other openings can help increase the rate at which bodily liquids can move through the thickness of the topsheet layer and penetrate into the other components of the article (e.g. into the absorbent structure 30). The selected arrangement of liquid-permeability is desirably present at least on an operative portion of the topsheet layer that is appointed for placement on the body-side of the article. The topsheet layer 26 can provide comfort and conformability, and can function to direct bodily exudates away from the body and toward the absorbent structure 30. In a desired feature, the topsheet layer 26 can be configured to retain little or no liquid in its structure, and can be configured to provide a relatively comfortable and non-irritating surface next to the body-tissues of a female wearer. The topsheet layer 26 can be constructed of any material which is also easily penetrated by bodily fluids that contact the surface of the topsheet layer.

The topsheet 26 can also have at least a portion of its bodyside surface treated with a surfactant to render the topsheet more hydrophilic. The surfactant can permit arriving bodily liquids to more readily penetrate the topsheet layer. The surfactant may also diminish the likelihood that the arriving bodily fluids, such as menstrual fluid, will flow off the topsheet layer rather than penetrate through the topsheet layer into other components of the article (e.g. into the absorbent body structure). In a particular configuration, the surfactant can be substantially evenly distributed across at least a portion of the upper, bodyside surface of the topsheet 26 that overlays the upper, bodyside surface of the absorbent.

The topsheet 26 may be maintained in secured relation with the backsheet 28 and/or the absorbent structure 30 by bonding all or a portion of the adjacent surfaces to one another. A variety of bonding techniques known to one of skill in the art may be utilized to achieve any such secured relation. Examples of such techniques include, but are not limited to, the application of adhesives in a variety of patterns between the two adjoining surfaces, entangling at least portions of the adjacent surface of the absorbent with portions of the adjacent surface of the topsheet, or fusing at least portions of the adjacent surface of the topsheet to portions of the adjacent surface of the backsheet and/or absorbent.

The topsheet 26 extends over the upper, bodyside surface of the article, and typically extends over any employed absorbent structure to provide a bodyside liner. The topsheet can optionally extend around the article to partially or entirely, surround or enclose the absorbent structure. Alternatively, the topsheet 26 and the backsheet 28 can have peripheral margins which extend outwardly beyond the terminal, peripheral edges of the absorbent structure 30, and the extending margins can be operatively joined or otherwise operatively connected together to partially or entirely, surround or enclose the absorbent structure.

The baffle or backsheet 28 may include a layer constructed of any operative material, and may or may not be configured to be liquid-permeable. In a particular configuration, the backsheet 28 may be configured to provide an operatively liquid-impermeable layer. The backsheet may, for example, include a polymeric film, a woven fabric, a nonwoven fabric or the like, as well as combinations or composites thereof. For example, the backsheet may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the backsheet 28 can operatively permit a sufficient passage of air and moisture vapor out of the article, particularly out of an absorbent (e.g. the storage or absorbent structure 30) while blocking the passage of bodily liquids. An example of a suitable backsheet material can include a breathable, microporous film, such as a HANJIN Breathable backsheet available from Hanjin Printing, Hanjin P&C Company Limited, a business having offices located in Sahvon-li.Jungan-mvu.Kongiu-City, Chung cheong nam-do, Republic of South Korea. The backsheet material is a breathable film, which is white in color, dimple embossed, and contains: 47.78% calcium carbonate, 2.22% TiO₂, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed cell polyolefin foam. For example, a closed cell polyethylene foam may be employed. Still another example of a backsheet material would be a material that is similar to a polyethylene film which is used on commercially sold KOTEX brand pantiliners. Suitable film materials have been obtainable from Pliant Corporation, a business having offices located in Schaumburg, Ill., USA.

The structure of any employed absorbent body 30 can be operatively configured to provide a desired level of absorbency or storage capacity. More particularly, the absorbent body can be configured to hold a liquid, such as urine, menses, other complex liquids or the like, as well as combinations thereof. As representatively shown, the absorbent body can include a matrix of absorbent fibers and/or absorbent particulate material, and the absorbent fiber can include natural and/or synthetic fiber.

Any employed absorbent structure 30 may also include superabsorbent material. Superabsorbent materials suitable for use in the present invention are known to those skilled in the art, and may be in any operative form, such as layers, fibers, particles or the like, as well as combinations thereof. Generally stated, the water-swellable, generally water-insoluble, hydrogel-forming polymeric absorbent material (superabsorbent) is capable of absorbing at least about 10, desirably about 20, and possibly about 100 times or more its weight in water. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company, Hoechst Celanese Corporation, Allied Colloid Inc., and Stockhausen, Inc.

The absorbent body 30 can be substantially unitary with a non-uniform structure or a generally uniform structure. Alternatively, the absorbent body may include a composite structure having a selected plurality of strata or layers. For example, the absorbent body structure may include an intake layer, a distribution layer, a transfer layer, a transfer-delay layer, a shaping layer, a retention layer or the like, as well as combinations thereof. The various strata and/or layers may be stacked, distributed or otherwise arranged in any operative sequence or configuration.

Additionally, the absorbent article can include any desired pattern or array of embossments. In particular aspects, the embossments may be formed on the bodyside surface of the article. Desired arrangements can include an absorbent body structure that has embossment regions formed on at least its bodyside surface. Similarly, the other employed components of the article can also include corresponding embossed regions.

The absorbent body 30 may, for example, include either or both of an intake layer, and an absorbent retention layer. The absorbent retention layer can be positioned between the topsheet 26 and the backsheet 28, and the intake layer can be positioned between topsheet 26 and the retention layer. The absorbent body can further include one or more additional layers positioned between the topsheet 26 and backsheet 28. The various individual layers may be separately provided layer-components, may be integrally formed together, or may be provided as any operative combination of separately-provided and integrally-formed layers.

The article 20 can include a system of side “wings” or wing-panel portions 42 which are positioned along both lateral side regions 64 of the article. The wing-panels can be separately provided members that are subsequently attached or otherwise operatively joined or operatively connected to the intermediate portion of the article 20. The separately provided wing-panel member can be operatively attached to at least one of the backsheet 28 and/or topsheet 26. In an alternative arrangement, each wing-panel 42 can be provided by an integrally-formed side portion of the article 20. The wing-panels may be integrally formed from another component of the article, such as the topsheet and/or the backsheet, and operatively joined or otherwise operatively connected to appointed sections of the article side regions 64 along the intermediate portion of the article.

In a particular configuration, each wing-panel 42 can include an integrally-formed side portion of the backsheet 28 which extends laterally past a corresponding, terminal side edge of the absorbent body 30. In another arrangement, each wing-panel 42 can include an integrally-formed side portion of the topsheet 26 which extends laterally past a corresponding, terminal side edge of the absorbent body. Still a further arrangement can have a configuration wherein each wing-panel 42 includes a panel composite. The panel composite can, for example, include an integrally-formed side portion of the backsheet 28 which extends laterally past a corresponding, terminal side edge of the absorbent body, and an integrally-formed cooperating, side portion of the topsheet 26 which extends laterally past the corresponding, terminal side edge of the absorbent body. The cooperating, side portion of the topsheet 26 is positioned in facing relation with the integrally-formed side portion of the backsheet 28.

In the cooperating pair of wing-panels, the first wing-panel 42 can be attached or otherwise operatively connected to a first side edge region 64 in the intermediate portion 76 of the article 20, and the second wing-panel 42a can be attached or otherwise operatively connected to a second side edge region 64a in the intermediate portion of the article. Each wing-panel 42 (42, 42a) can be configured to operatively wrap about an undergarment of a wearer.

The wing-panels can have an appointed storage position in which the wing-panels 42 are directed generally inwardly toward the longitudinally-extending centerline 52. The wing-panel that is connected to extend from one side margin may have sufficient cross-directional length to extend and continue past the centerline 52 to approach the laterally opposite side margin of the article. The storage position of the wing-panels can ordinarily represent an arrangement observed when the article is first removed from its wrapper or other packaging. Prior to placing the article into a bodyside of an undergarment prior to use, the wing-panels 42 can be selectively arranged to extend laterally from the side regions 64 of the article intermediate portion. After placing the article in the undergarment, the wing-panels 42 can be operatively wrapped and secured in an in-use position around the side edges of the undergarment crotch portion to help hold the article in place. Typically, the wing-panels are configured to secure the article to a crotch portion of the wearer's undergarment.

The wing-panel portions 42 can have any operative construction, and can include a layer of any operative material. Additionally, each wing-panel can comprise a laminate or other composite material. For example, the wing-panels may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

The wing-panel materials can be substantially non-stretchable or may be stretchable. The wing-panel material may also be capable of providing a selected amount of elastomeric stretch and retraction. In particular configurations, the wing-panel material can exhibit a maximum stretch elongation value of up to about 300%, or more. In other configurations, the wing-panel material can exhibit a minimum stretch elongation value of 50%. In other features, the wing-panel material can have a basis weight which is within the range of about 0.5-3 ounces per square yard (about 17-102 g/m²). By employing such wing-panel materials, the wing-panel can help provide desired fit characteristics, and can help provide improved leak protection.

Each wing-panel 42 can include a panel-fastener component 44 which is operatively joined or otherwise operatively connected to a major facing surface of the associated wing-panel. Such major surface is typically appointed to engage and become operatively attached to an outward-facing surface of the wearer's undergarment. The panel-fastener 44 can include any operative fastener component, such as a component of an interengaging mechanical faster, an adhesive fastener, a cohesive fastener, a magnetic fastener, an electromechanical fastener or the like, as well as combinations thereof. The mechanical fastener component may, for example, include a suitable hook component, such as a J-hook, mushroom-head hook, flat-top nail-head hook, a palm-tree hook, a multiple-J hook or the like, as well as combinations thereof. Optionally, the mechanical fastener component may, for example, include a suitable loop component. The loop component may, for example, include a knit fabric, a woven fabric, a nonwoven fabric or the like, as well as combinations thereof.

Each wing-panel 42 can include a panel base section and a panel free-end region. Each panel base section can be operatively joined or otherwise operatively connected to its corresponding side edge region 64 of the article 20, and each panel free-end region can extend from its corresponding panel base section. The free-end region can be further configured to operatively wrap around an appointed region of the wearer's undergarment. For example, the free-end regions of the wing-panels can be wrapped about a crotch region of the wearer's undergarment.

With reference to FIGS. 2 and 3, a selected configuration of a garment-fastener or other garment-attachment mechanism 38 may be operatively distributed and operatively joined or otherwise operatively connected onto the garment-side surface of the article 20 to help secure the article to the undergarment. The garment-fastener can include any operative fastener mechanism, such as a component of an interengaging mechanical fastener, an adhesive fastener, a cohesive fastener, a magnetic fastener, and electromechanical fastener or the like, as well as combinations thereof. For example, the garment-fastener can be provided by the representatively shown adhesive 38a, and the garment-fastener may be arranged in any operative pattern, such as one or more strip regions that are distributed onto the garment-side of the article. In a particular arrangement, an operative garment adhesive can be distributed over the garment-side of the backsheet, and one or more layers or sheets of release material 40 can be removably placed over the garment adhesive during storage prior to use. In desired arrangements, at least a significant portion of the garment-fastener 38 can be generally aligned with or otherwise located along the longitudinal centerline 52 of the article.

In the construction of the article 20, the various components may be assembled and held together with any operative securement mechanism or system. For example, the desired attachments or securements can include adhesive bonds, cohesive bonds, thermal bonds, ultrasonic bonds, pins, snaps, staples, rivets, stitches, welds, zippers, or the like, as well as combinations thereof.

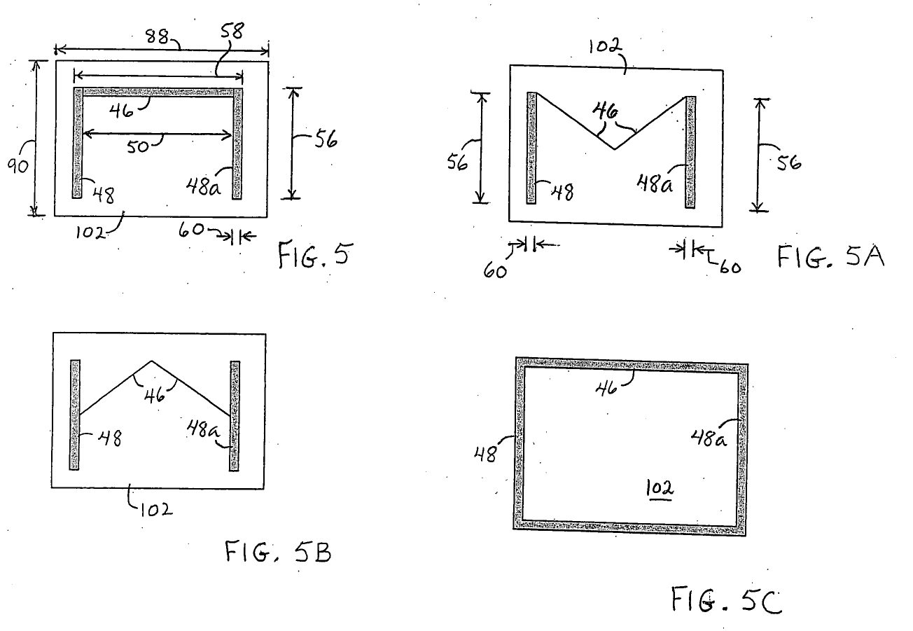

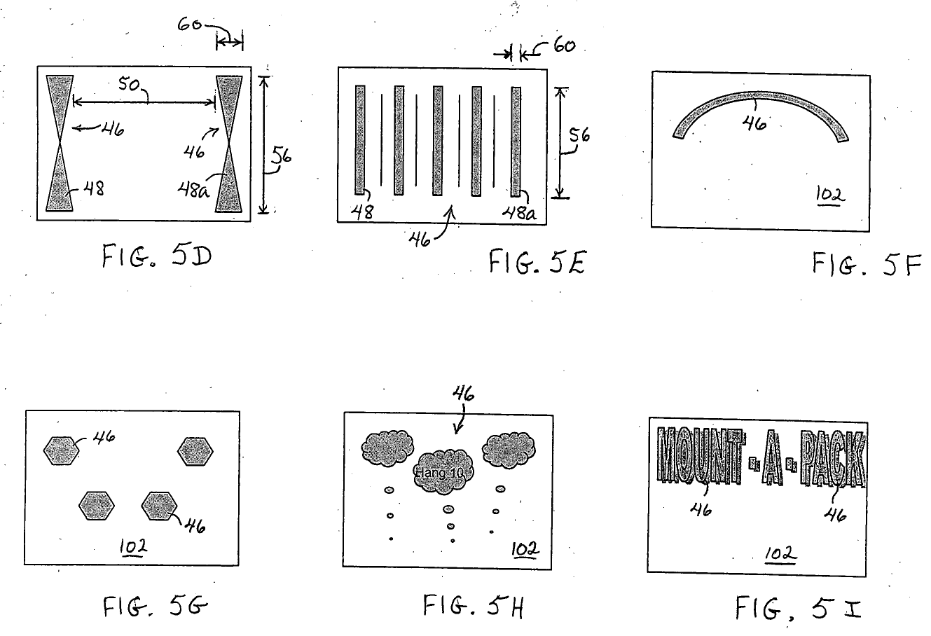

With reference again to FIGS. 1, 1A and 5 through 5I, the package 82 can have any operative size, shape or configuration appropriate for holding and containing the selected articles. The package can desirably be a disposable or other limited-use package, which is intended to be discarded when emptied of product. The package may be substantially rigid or flexible, or may include an operative combination of flexible and substantially rigid portions. The substantially rigid package includes package wall panels which do not significantly deform or collapse under the weight of the package when the package 82 is substantially empty of the articles 20 and placed on a substantially flat, horizontal support surface. Examples of such packages can be provided by cartons, boxes, polymer shells and the like, as well as combinations thereof.

In contrast, the flexible package includes wall panels which can operatively deform or collapse under the weight of the package when the package is substantially empty of the articles and placed on the substantially flat, horizontal surface. Examples of such flexible packages can be provided by a soft-sided package with wall panels made with films, woven or nonwoven fabrics, mesh materials or the like, as well as combinations thereof. Additionally, the package can include a package top and a package bottom. The package top and/or bottom can be configured to be flexible or substantially rigid, as desired. The package 82 can be configured to be sufficiently flexible to significantly collapse under its own weight when the empty package is placed on the horizontal support surface.

The selected mounting surface 74 can be provided by any operative, substantially vertically oriented surface. The mounting surface can, for example, be provided by a surface of a side wall of a room, a side-surface of a cabinet, a side-surface of an extending panel or the like. The mounting surface may be irregular or substantially smooth. Desirably, the selected mounting surface is sufficiently smooth to operatively engage a mounting mechanism 46 which is provided by an adhesive.

The mounting mechanism 46 can be provided by any operative device or system. For example, the mounting mechanism can include a component of an interengaging mechanical fastener system, a component of a hook-and-loop fastener, a system of suction devices, a sticky-tack low temp wax, a system of cohesive materials, an adhesive tape, a spray-on adhesive, a low-tack or pressure-sensitive adhesive and the like, as well as combinations thereof. In a desired arrangement, the mounting mechanism can include a releasable adhesive, and the releasable adhesive can be pressure-sensitive. Suitable adhesives are well known and are available from commercial vendors, such as National Starch and Chemical Company, a business having offices located in Bridgewater, N.J., U.S.A.; and Bostik-Findley, a business having offices located in Wauwatosa, Wis., U.S.A. Examples of suitable releasable adhesives can include a 34-5610 hot melt, a HL 8151 hot melt, EASYMELT 34-5602, #570-5504, and DISPOMELT 34-5604 adhesives.

Various types of well known, repositionable pressure sensitive adhesives (RPSAs) can be used in the present invention. The RPSAs can be removable and repositionable. In this context, the term “repositionable” means that the adhesive in a conventional way can be adhered to and removed from a clean solid surface at least two times without substantially losing tack. Preferably, the sheet can be adhered to and removed from a clean solid surface at least ten times, and more preferably, at least twenty times, without substantially losing tack. RPSAs are well known in the art as evidenced by U.S. Pat. Nos. 5,045,569; 4,988,567; 4,994,322; 4,786,696; 4,166,152; 3,857,731; and 3,691,140, the disclosures of which are incorporated here by reference.

In a particular arrangement, the releasable adhesive may be provided by the adhesive system employed with the CORD CLIPS WITH COMMAND ADHESIVE product, part number 17017, which is manufactured by 3M Construction and Home Improvement Markets Division, a business having offices located in St. Paul, Minn., U.S.A. The employed system has an extending adhesive portion that can be grasped and pulled by the consumer to break the adhesive attachment.

The mounting mechanism can desirably be a limited use mechanism, and the limited-use mounting mechanism is typically discarded when the package is emptied of articles. The mounting mechanism desirably has an attachment strength that is sufficiently high to operatively hold the package on the desired mounting surface, while also being sufficiently limited to enable a desired removal of the package from the selected mounting surface without excessively marring the surface. In a desired arrangement, the package 82 can be a soft-sided package formed from flexible materials, and the mounting mechanism can, for example, be provided by an operative, low-tack adhesive configured to reliably hold the package on the selected mounting surface, such as a surface provided on the inside of a cabinet door or other storage area. As a result, the storage system of the invention can be easier to find and store. Additionally, the mounting mechanism can be configured to improve the operation of the soft-sided package. The mounting mechanism can be configured to more effectively hold up the laterally-opposed side portions of a mounting panel of the soft-sided package, and to more effectively present the contained articles for easy access even when the package is only partially filled with the contained articles.

The mounting mechanism 46 is operatively connected and secured to an appointed mounting panel 102 of the package 82. With a soft-sided package, the mounting mechanism 46 can be sprayed on, or otherwise attached to the package 82 prior to use. Desirably, the mounting mechanism can be secured to an outwardly-facing surface of the package with a factory-attachment that is generated in a factory-process during the construction of the package. In a particular arrangement, the mounting mechanism could be positioned below the outermost surface of the package. For example, the package may include an outermost layer which includes an open-weave, nonwoven material, and an adhesive layer can be located below the outermost surface of the open-weave material. When a person presses the package to the wall, the adhesive can be operatively moved past the nonwoven layer to attach to the selected mounting surface. As a result, there may be less need for providing a protective cover over the adhesive before use.

In another aspect, the mounting mechanism 46 can be configured to operatively suspend a total weight of the package 82 and its contained articles 20. The package 82 can be rather large and bulky, and can contain a relatively large quantity of products. As a result, the combined weight of the package and contained articles can pull the mounting mechanism away from the attachment area against the selected mounting surface. The pulling force can come from the vertical direction or the horizontal direction.

The selected attachment strength, and the selected distribution and orientation of the attachment area provided by the mounting mechanism 46 can help increase the length of time over which the provided mounting attachment can reliably hold the package on the selected mounting surface. The attachment strength and the selected distribution and orientation of the attachment area provided by the mounting mechanism can also help hold the package in a desired upright position, and can allow an easier access to the contained articles after the package is opened.

The mounting panel 102 of the package 82 can have a selected size and shape. Typically, the mounting panel has a panel width 88 and a panel height 90. In particular aspects, the mounting panel area can be at least a minimum of about 1 in² (about 6.5 cm²). The mounting panel area can alternatively be at least about 4 in² (about 26 cm²), and can optionally be at least about 9 in² (about 58 cm²) to provide desired benefits. In other aspects, the mounting panel area can be up to a maximum of about 324 in² (about 2090 cm²), or more. The mounting panel area can alternatively be up to about 210 in² (about 1355 cm²), and can optionally be up to about 132 in² (about 852 cm²) to provide desired effectiveness.

The mounting mechanism 46 can be configured to provide a distinctively arranged mounting-attachment area. The attachment area can have any operative size and shape, and can be oriented along any operative direction or combination of directions. In a particular aspect, the mounting mechanism 46 can provide a mounting-attachment area which is at least a minimum of about 0.125 in² (about 0.8 cm). The mounting-attachment area can alternatively be at least about 2 in² (about 13 cm), and can optionally be at least about 6 in² (about 39 cm) to provide desired performance. In other aspects, the mounting-attachment area can be up to a maximum of about 306 in² (about 1974 cm), or more. The mounting-attachment area can alternatively be up to about 105 in² (about 677 cm), and can optionally be up to about 88 in² (about 568 cm) to provide desired benefits. In a desired arrangement, the mounting-attachment area can be about 4.5 in² (about 29 cm²).

In another aspect, the desired mounting-attachment area can be distributed along and divided between two lateral portions 48, 48a of the mounting mechanism 46, and each of the lateral portions can be configured to provide a user-attachment area of at least a minimum of about 1 inch² (about 6.5 cm²). Each of the lateral portions can have a user-attachment area which is alternatively at least about 8 cm², and is optionally at least about 10 cm² to provide desired performance. Additionally, each of the lateral portions can have a user-attachment area which is up to about 200 cm² or more. In a further aspect, the desired mounting-attachment area can be distributed along and divided between two or more, vertically aligned strip regions of the mounting mechanism.

The attachment-area, as well as selected portions of the attachment-area, can have a corresponding width and a corresponding height. In a particular aspect, at least a significant portion of the mounting attachment area can be positioned to extend primarily along the vertical direction, with the width of the attachment area configured to be significantly smaller than its height. It has been found that when the mounting mechanism is configured to provide an attachment area that is positioned to extend primarily along only the horizontal direction, with the width of the mounting attachment area significantly greater than its height, the package would prematurely separate from the mounting surface after approximately one day. In contrast, by using the same amount of attachment area but positioning it to extend primarily along the vertical direction, with the width of the attachment area significantly smaller than its height, the package could then stay attached to the mounting surface for a significantly longer period of time, until removal was desired. While not intending to be bound by any particular theory, it is believed that the forces that tended to detach the package from the mounting surface were advantageously redistributed with the change in orientation and distribution of the significant amounts of the more vertically distributed attachment areas provided by the mounting mechanism.

In a desired feature, a configuring of predetermined portions of the mounting mechanism 46 at laterally opposing regions of the package can help keep the package exit mechanism 84 readily available for use. In another feature, at least a selected portion of the mounting mechanism can be configured to provide an operative section of the mounting attachment area that extends substantially continuously along the horizontal, lateral direction of the package. The laterally extending section of the mounting attachment area can be located proximate top of the package to help hold open the package. In still other arrangements, the mounting mechanism can include a combination of the vertically extending portions and horizontally extending portions. As a result, the exit mechanism 84 of the package can be more reliably mounted and can more readily stay open.

As representatively shown in the examples illustrated in FIGS. 1A and 5, the mounting mechanism can include first and second, laterally-distal portions (48, 48a) which are laterally spaced apart by a spacing distance 50. In a particular aspect, the spacing distance can be at least a minimum of about 5% of the mounting panel 102 of the package. The spacing distance can alternatively be at least about 15% of the mounting panel, and can optionally be at least about 33% of the mounting panel to provide desired benefits. In other aspects, the spacing distance 50 can be up to a maximum of about 97% of the mounting panel, or more. The spacing distance can alternatively be up to about 95% of the mounting panel, and can optionally be up to about 90% of the mounting panel to provide desired effectiveness.

In a further aspect the spacing distance 50 can be at least a minimum of about 0.125 inch (about 0.32 cm). The spacing distance can alternatively be at least about 1 inch (about 2.54 cm) or about 1.7 inch (about 4.3 cm), and can optionally be at least about 2 inch (about 5.1 cm) or about 3 inch (about 7.6 cm) to provide desired benefits. In other aspects, the spacing distance 50 can be up to a maximum of about 17.5 inch (about 44 cm), or more. The spacing distance can alternatively be up to about 17 inch (about 43 cm), and can optionally be up to about 16 inch (about 41 cm) to provide desired performance.

If the spacing distance 50 is outside the desired values, the bag may excessively sag, and it may be excessively difficult to access the articles within the package. Additionally, an undesired amount of the mounting mechanism may be exposed to contamination.

Another aspect of the invention can include a configuration in which at least one significant, vertically-elongated portion of the mounting mechanism 46, particularly a vertically-elongated portion of its mounting-attachment area, extends over a distinctive longitudinal length 56. In a desired arrangement, each of a plurality of the vertically-elongated portions of the mounting mechanism 46 can extend over the longitudinal length 56. In particular configurations, for example, each of the lateral portions (48, 48a) of the mounting mechanism can be vertically-elongated to have the selected longitudinal length 56. Optionally, one or more middle or medial portions of the mounting mechanism can be configured to have the elongated longitudinal length.

In particular aspects, the longitudinal length 46 can be at least a minimum of about 67% or 70% of the vertical height 90 of the mounting panel 102 of the package. The longitudinal length can alternatively be at least about 75% of the mounting panel height, and can optionally be at least about 80% of the mounting panel height to provide desired benefits. In other aspects, the longitudinal length 46 can be up to a maximum of about 100% of the height 90 of the mounting panel, or more. The longitudinal length can alternatively be up to about 95% of the mounting panel height, and can optionally be up to about 90% of the mounting panel height to provide desired effectiveness.

In further aspects, the longitudinal length of the mounting mechanism can be at least a minimum of about 1 inch (about 0.32 cm) or about 1.5 inch (about 2.54 cm). The longitudinal length can alternatively be at least about 1.7 inch (about 4.3 cm), and can optionally be at least about 2 inch (about 5.1 cm) or about 3 inch (about 7.6 cm) to provide desired performance. In other aspects, the longitudinal length 56 can be up to a maximum of about 17.5 inch (about 44 cm), or more. The longitudinal length of the mounting mechanism can alternatively be up to about 16 inch (about 43 cm), and can optionally be up to about 16 inch (about 41 cm) to provide improved desired benefits. In a particular configuration, for example, the longitudinal length 56 of the mounting mechanism can be about 4.5 inch (about 11.4 cm).

If the longitudinal length 56 is outside the desired values, there may be inadequate attachment between the package and the selected mounting surface. Accordingly, the bag may prematurely detach from the vertical mounting surface. Additionally, an undesired amount of the mounting mechanism may be exposed to undesired contamination.

In a further aspect, the mounting mechanism or a selected portion thereof can provide at least one, significant portion of attachment area that has a high, vertical aspect ratio. Where the attachment-area of the selected portion of the mounting mechanism has an area-portion A (cm²); and a vertically oriented height dimension h (cm), such as provided by a longitudinal length 56 of the mounting mechanism; the vertical aspect ratio of the attachment-area portion can be determined by the calculation: h²/A. In a particular arrangement, for example, each of the lateral portions (48, 48a) of the mounting mechanism can be configured to have the high, vertical aspect ratio. Optionally, a middle or medial portion of the mounting mechanism can be configured to have the high, vertical aspect ratio. The vertical aspect ratio of the selected mounting area portion can be at least a minimum of about 4. The aspect ratio can alternatively be at least about 7, and can optionally be at least about 10 or at least about 15 to provide improved benefits. In desired arrangements, for example, the vertical aspect ratio can be 20 or more.

If the vertical aspect ratio is outside the desired values, there may be an insufficient level of attachment between the package and the selected mounting surface. Accordingly, the bag may prematurely detach from the vertical mounting surface.

In a further aspect, at least a portion of the mounting mechanism 46 can be configured to be noncontiguous or substantially contiguous along a lateral, width direction of the package 82. In a desired configuration, at least a significant portion of the mounting mechanism 46 can be configured to be noncontiguous along the lateral, width direction of the package.

With reference to FIGS. 5 through 5C and 5F, for example, at least a portion of the mounting mechanism 46 may be configured to be substantially contiguous along a lateral width distance (e.g. width distance 58) which is at least a minimum of about 2% or 5% of the overall width 88 of the package 82. The mounting mechanism can alternatively be configured to be substantially contiguous along a lateral width distance which is at least a minimum of about 10% of the overall package width 88, and can optionally be configured to be substantially contiguous along a lateral width distance which is at least about 15% of the overall package width to provide desired performance. In another aspect, the mounting mechanism can be substantially contiguous along a lateral width distance which is up to a maximum of about 95% or 100% of the overall width of the package to provide desired benefits.

Another aspect of the invention can have a configuration in which at least a portion of the mounting mechanism 46 is configured to be substantially contiguous along a lateral, width distance 58 which is at least a minimum of about 0.25 inch (about 0.64 cm). The mounting mechanism can alternatively be substantially contiguous along a lateral, width distance which is at least about 1 inch (about 2.54 cm), and can optionally be substantially contiguous along a lateral, width distance which is at least about 1.5 inch (about 3.8 cm) to provide improved [desired] benefits. In other aspects, the mounting mechanism 46 can be substantially contiguous along a lateral, width distance which is up to a maximum of about 18 inch (about 46 cm), or more to provide desired benefits.

If the contiguous width of the mounting mechanism is outside the desired values, the bag may not adequately remain open. Accordingly, it may be excessively difficult to readily access the remaining articles within the package.

With reference to FIGS. 5 through 5I, the mounting mechanism can optionally be configured to provide a mounting attachment area that is distributed in various arrangements and patterns. Various sections of the mounting mechanism can be noncontiguous or substantially contiguous with one another, and can have any desired shape. For example, sections of the mounting mechanism may have the shapes of text, characters, visual graphics, geometrical shapes or the like, as well as combinations thereof.

In a desired arrangement, for example, the mounting mechanism 46 can include a first section 48, and at least a second section 48a that is laterally spaced from the first section. The mounting mechanism may or may not include a contiguous portion that extends between the spaced apart sections. As representatively shown, the first section 48 can, for example, have the configuration of a first stripe that is substantially vertically aligned, and the second section can have the configuration of a second stripe that is substantially vertically aligned.

In a particular feature, the first and/or second sections 48, 48a can have a lateral width 60 which is at least a minimum of about 0.3 cm. The lateral width 60 can alternatively be at least about 0.6 cm, and can optionally be at least about 1 cm to provide desired benefits. In a further feature, the first and/or second sections 48, 48a can have a lateral width 60 which is up to a maximum of about 3 cm or more. The lateral width 60 can alternatively be up to about 2 cm, and can optionally be up to about 1.5 cm to provide desired benefits. In a desired arrangement, for example, the lateral width can be about 0.5 inch (about 1.27 cm).

In another aspect, the package 82 can be configured to include a guard mechanism which covers or otherwise protects the mounting mechanism prior to use by the consumer. The guard mechanism can be particularly helpful when the mounting mechanism includes an adhesive. The guard mechanism can, for example, include a cover layer or other extending member which operatively overlies the adhesive or other mounting mechanism. The cover layer may be a separately provided component, or may be provided by a portion of an existing component of the storage system. Additionally, the cover layer may be substantially blank, or may include any desired graphics and/or text.

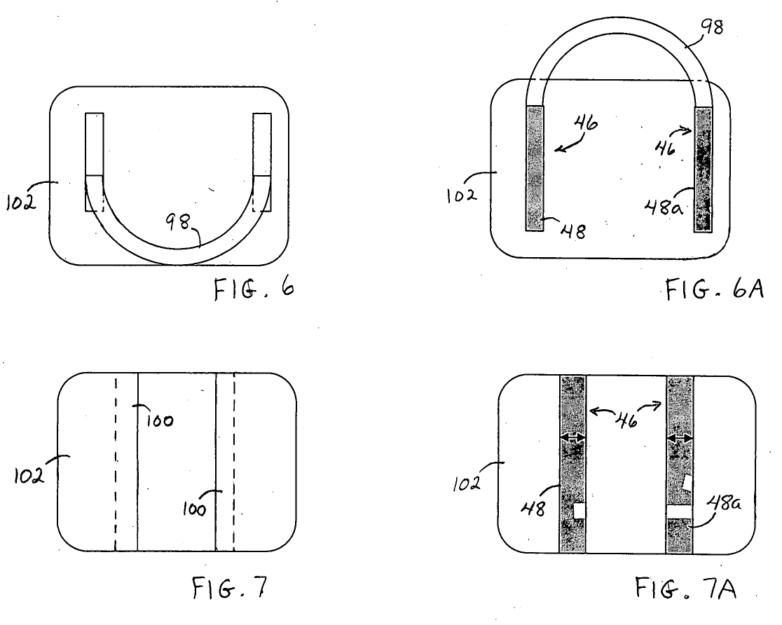

As illustrated in FIGS. 6 and 6A, for example, the package 82 can include a handle member 98. Additionally, the handle member can be movable from a guard position to a user position. In the guard position, the material of the handle member can operatively cover and releasably attach to the mounting mechanism 46. The handle member can be readily detached from the mounting mechanism to expose the mounting mechanism, and moved to the desired user-position.

With reference to FIGS. 7 and 7A, the package 82 can include a panel that includes at least one pleated fold 100. As illustrated, the package can include a cooperating plurality of pleated folds, and the pleats may be arranged to extend generally vertically along the longitudinal direction of the package. In the guard position, the pleats are closed, and the pleat section can extend over and cover their corresponding sections of the mounting mechanism. In the user position, the pleats can be operatively expanded and moved to an open position to expose their corresponding sections of the mounting mechanism.

The exit mechanism 84 can be configured for location on a selected package panel 96, which may be a top panel, a side panel or a bottom panel, as desired. Any operative exit mechanism can be employed, as desired. With reference to FIGS. 8 and 8A, for example, the package 82 may include an opening lid or flap 66 having a fixed edge region 68 and a distal, movable edge region 70. In another feature, the flap 66 can be positioned to extend over an upper, top region of the package 82. The selected package panel can include or otherwise provide a package opening 86 having a boundary edge. The package opening can have any appropriate size and shape that allows a convenient access to the contained articles 20. For example, the package opening may be rectilinear, curvilinear, round, oval and the like, as well as combinations thereof.

As representatively shown, a portion 47 of the mounting mechanism 46 can be located on the flap 66 and can be configured to operatively attach to the appointed mounting surface 74 to selectively hold a flap 66 in an open position. In another feature, the exit mechanism 84 may include a user-actuated closure mechanism 94 which can re-close the exit mechanism 84 to a position which operatively covers the package opening 86 (e.g. FIG. 1). In an optional configuration, the closure mechanism can be configured to selectively and releasably attach to the vertical mounting surface 74.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. An article storage system, comprising: a package; a plurality of personal care articles contained in the package; and a mounting mechanism which is configured to releasably attach the package to a generally vertical mounting surface; wherein
the mounting mechanism is secured to the package;
the mounting mechanism includes first and second, distal, lateral-portions which are laterally spaced apart by a spacing distance;
the spacing distance is at least a minimum of about 5% of an overall width of the package when filled with the articles;
the package includes an exit mechanism which is configured to provide a package opening through which the articles can be removed from the package.

2. A storage system as recited in claim 1, wherein the mounting mechanism has been secured to an outwardly-facing surface of the package with a substantially permanent attachment

3. A storage system as recited in claim 1, wherein the mounting mechanism has been secured to an outwardly-facing surface of the package with a factory-attachment.

4. A storage system as recited in claim 1, wherein the spacing distance is at least about 15% of the overall width of the package.

5. A storage system as recited in claim 1, wherein each of the lateral-portions of the mounting mechanism extends over a longitudinal length which is at least about 67% of a vertical height of the package.

6. A storage system as recited in claim 1, wherein each of the lateral portions of the mounting mechanism is configured to provide a user-attachment area of at least a minimum of about 6.5 cm².

7. A storage system as recited in claim 1, wherein each of the lateral portions of the mounting mechanism are configured to have a vertical aspect ratio of at least a minimum of about 4.

8. A storage system as recited in claim 1, wherein the package is configured to operatively deform under its own weight when the package is substantially empty of the articles.

9. A storage system as recited in claim 8, wherein the package is configured to be disposable.

10. A storage system as recited in claim 1, wherein the mounting mechanism is configured to operatively suspend a total weight of the package and its contained articles.

11. A storage system as recited in claim 1, wherein at least a portion of the mounting mechanism is configured to be substantially contiguous along at least a minimum of about 2% the overall width of the package.

12. A storage system as recited in claim 1, wherein the exit mechanism includes an opening flap having a fixed edge region and a distal, movable edge region.

13. A storage system as recited in claim 11, wherein a portion of the mounting mechanism is located on the flap and configured to operatively attach to the mounting surface to selectively hold the flap in an open position.

14. A storage system as recited in claim 1, further including a movable article retention member which is located within the package and is positioned above the articles.

15. A storage system as recited in claim 1, wherein
the mounting mechanism includes a first section, and at least a second section that is laterally spaced from the first section;
the first section has the configuration of a first stripe that is substantially vertically aligned; and
the second section has the configuration of a second stripe that is substantially vertically aligned.

16. A storage system as recited in claim 1, wherein the mounting mechanism includes a releasable adhesive.

17. A storage system as recited in claim 1, wherein each article includes a liquid-permeable topsheet and a backsheet operatively connected to the topsheet.

18. A storage system as recited in claim 1, wherein each article includes an absorbent body sandwiched between its corresponding topsheet and backsheet.

19. A storage system as recited in claim 1, wherein each article further includes a garment-attachment mechanism which is secured to the backsheet of its corresponding article.
